(19) United States
(12) Patent Application Publication
(10) Pub. No.: US 2024/0042085 A1
(43) Pub. Date: Feb. 8, 2024

(54) METHODS AND SYSTEMS FOR DISPENSING A VOLATILE MATERIAL

(71) Applicant: S. C. JOHNSON & SON, INC., Racine, WI (US)

(72) Inventors: Sebastian D. HASIK, Antioch, IL (US); Thomas P. GASPER, Germantown, WI (US)

(21) Appl. No.: 18/229,043

(22) Filed: Aug. 1, 2023

Related U.S. Application Data
(60) Provisional application No. 63/395,218, filed on Aug. 4, 2022.
(60) Provisional application No. 63/435,494, filed on Dec. 27, 2022.

(51) Int. Cl. A61L 9/03

(52) U.S. Cl. CPC A61L 9/032; A61L 9/035; A61L 2209/111

(57) ABSTRACT

A method of emitting a volatile material from a volatile material dispenser includes the steps of identifying a first vapor pressure of a first fragrance characteristic of the volatile material, identifying a second vapor pressure of a second fragrance characteristic of the volatile material that is different than the first vapor pressure, heating the volatile material to a first temperature associated with the first fragrance characteristic, and heating the volatile material to a second temperature associated with the second fragrance characteristic that is different than the first temperature.

### CROSS REFERENCE TO RELATED APPLICATIONS

This application is claims priority benefit of, and incorporates herein by reference for all purposes, U.S. Provisional Patent Applications 63/395,218 filed Aug. 4, 2022 and 63/435,494 filed Dec. 27, 2022.

### BACKGROUND OF THE DISCLOSURE

### 1. Field of the Disclosure

The present disclosure relates generally to methods and systems for dispensing volatile materials, and more particularly, to systems and methods for volatizing a liquid containing a mixture of fragrances to achieve varying fragrance characteristics to customize and improve user control and experience.

### 2. Description of the Background of the Disclosure

A multitude of volatile material diffusion devices or diffusers exist in the marketplace. Many of such devices are passive devices that require only ambient air flow to disperse the liquid active material therein. Other devices are battery-powered or receive household power via a plug extending from the device. Some known diffusers include a heating element for heating a volatile material to promote vaporization thereof. Other diffusers employ a fan or blower to generate air flow to direct volatile material out of the diffuser into the surrounding environment. Still other diffusers that dispense volatile materials utilize ultrasonic means to dispense the volatile materials therefrom. Fragrance compositions that are used in the aforementioned volatile material dispensers are composed of a mixture of volatile perfume raw materials, and it is not uncommon for a fragrance composition to be composed of over twenty different perfume raw materials. The chemical properties of perfume raw materials in a fragrance composition often vary widely in terms of polarity, density, vapor pressure, flash point, and other properties.

A problem with past volatile material diffusers is that a user may become accustomed to or habituated to a particular volatile material, i.e., the perception by a user of a dispensed fragrance at a constant intensity tends to decay over time, and various diffusers have attempted to alleviate this problem. Some diffusers include a switch or other mechanism that is controlled by the user, whereby the user can change the intensity level at which the volatile material is dispensed, i.e., by adjusting a timing sequence of release. Still other diffusers include at least two fragrances that are emitted in an alternating sequence. However, emitting two different fragrances from a volatile material dispenser requires multiple fragrance cartridges and a more complicated dispensing system, e.g., multiple heaters or fans, which can be cost prohibitive.

Therefore, systems and methods for dispensing a volatile material that provide for customizable user control and experience, and that assist with the problem of habituation, but that do not require additional refill fragrance cartridges would be useful.

### SUMMARY OF THE DISCLOSURE

According to some aspects of the disclosure, a method of emitting a volatile material from a volatile material dispenser comprises the steps of identifying a first vapor pressure of a first fragrance characteristic of the volatile material, identifying a second vapor pressure of a second fragrance characteristic of the volatile material that is different than the first vapor pressure, heating the volatile material to a first temperature associated with the first fragrance characteristic, and heating the volatile material to a second temperature associated with the second fragrance characteristic that is different than the first temperature. The method further comprises the step of heating the volatile material to the first temperature and the second temperature using pulse with modulation. The method further includes the step of heating the volatile material to the first temperature for a first amount of time and the second temperature to a second amount of time that is different than the first amount of time. In some embodiments, the first fragrance characteristic is associated with a first composition that is not a fragrance, and the second fragrance characteristic is associated with a second composition that is a fragrance.

In some embodiments, a method of emitting a volatile material from a volatile material dispenser includes the step of causing air to flow out of the volatile material dispenser by an air displacement mechanism. In some embodiments, the air displacement mechanism is an air pump. In some embodiments, the air displacement mechanism is a fan. The method further includes the step of controlling a speed of the air placement mechanism to achieve varied fragrance intensities.

In some embodiments, a method of emitting a volatile material from a first material dispenser comprises the steps of providing a refill containing a volatile material, heating the volatile material to a first temperature associated with a first fragrance characteristic, heating the volatile material to a second temperature associated with a second fragrance characteristic, and heating the volatile material to a third temperature associated with a third fragrance characteristic. The first temperature is less than the second temperature, and the second temperature is less than the first temperature, and the first fragrance characteristic, the second fragrance characteristic, and the third fragrance characteristic are different from one another. In some embodiments, the first fragrance characteristic, the second fragrance characteristic, and the third fragrance characteristic are identified based on one or more chemical characteristics of the volatile material. In some embodiments the one or more chemical characteristics include a vapor pressure. In some embodiments, the first fragrance characteristic is associated with a first wattage, the second fragrance characteristic is associated with a second wattage, and the third fragrance characteristic is associated with a third wattage. The first wattage, the second wattage, and the third wattage are different from one another. In some embodiments, the first wattage, the second wattage, and the third wattage are between about 4.0 W and about 6.0 W. In some embodiments, the first wattage, the second wattage, and the third wattage are between about 1.0 W and about 9.0 W.

In some embodiments, a method for emitting a volatile material within a refill from a volatile material dispenser comprises heating the volatile material to a first temperature, controlling an intensity of a fragrance of a volatile material using a first switch, controlling a fragrance characteristic of the volatile material using a second switch that is configured to adjust the heat applied to the volatile material to a second temperature that is different than the first temperature, and receiving information from one or more information sources to regulate the volatile material dispenser between the first temperature and the second temperature. In some embodiments, the method can further include a third switch that is a push button on/off switch.

In some embodiments, the first switch has a first switch setting associated with a first timing between each operational cycle, a second switch setting associated with a second timing between each operational cycle, and a third switch setting associated with a third timing between each operational cycle. The first timing, the second timing, and the third timing are different from one another. In some embodiments, the first switch is provided via a display screen of an electronic device that is in communication with electrical components within the volatile material dispenser.

In some embodiments, the second switch has a first switch setting associated with a first switch setting defining a first fragrance characteristic, a second switch setting defining a second fragrance characteristic, and a third switch setting defining a third fragrance characteristic. The first fragrance characteristic is associated with a first power input, the second fragrance characteristic is associated with a second power input, and the third fragrance characteristic is associated with a third power input. The first power input, the second power input, and the third power input are different from one another. In some embodiments, the second switch is provided via a display screen of an electronic device that is in communication with electrical components within the volatile material dispenser.

### BRIEF DESCRIPTION OF THE DRAWINGS

FIG. 1 is an isometric view of a first embodiment of a volatile material dispenser as disclosed herein;

FIG. 2 is a left side view of the volatile material dispenser of FIG. 1;

FIG. 3 is a front view of the volatile material dispenser of FIG. 1;

FIG. 4 is a cross-sectional view of the volatile material dispenser taken through line 4-4 of FIG. 1;

FIG. 5 is a is a cross-sectional view of the volatile material dispenser taken through line 5-5 of FIG. 1;

FIG. 6 is an isometric view of the volatile material dispenser of FIG. 1 with a cover removed for clarity;

FIG. 7 is a first graph illustrating varying fragrance characteristics of a first fragrance and a second fragrance based on a power input or temperature of a heater of the volatile material dispensers disclosed herein;

FIG. 8 is a graph illustrating varying fragrance characteristics of the first fragrance, the second fragrance, and a third fragrance based on a power input or temperature of a heater of the volatile material dispensers disclosed herein;

FIG. 9 is an isometric view of another embodiment of the volatile material dispenser as disclosed herein;

FIG. 10 is a front, detail view of a lower end of the volatile material dispenser of FIG. 9;

FIG. 11 is a right side, detail view of the lower end of the volatile material dispenser of FIG. 9;

FIG. 12 is a rear view of the volatile material dispenser of FIG. 9;

FIG. 13 is a bottom view of the volatile material dispenser of FIG. 9 with a lower housing or base removed for clarity;

FIG. 14 is a perspective view of the base or lower housing of the volatile material dispenser of FIG. 9;

FIG. 15 is a top view of the volatile material dispenser of FIG. 9;

FIG. 16 is a bottom view of the volatile material dispenser of FIG. 9;

FIG. 17 is a partial perspective view of the volatile material dispenser of FIG. 9 shown with the base or lower housing separated from an upper housing for clarity;

FIG. 18 is an electrical schematic of an electrical assembly of the volatile material dispensers disclosed herein;

FIG. 19 is an electrical schematic of a user input stage of the electrical assembly of FIG. 18;

FIG. 20 is an electrical schematic of a heating stage of the electrical assembly of FIG. 18;

FIG. 21 is an electrical schematic of a controller stage of the electrical assembly of FIG. 18;

FIG. 22 is an electrical schematic of a power stage of the electrical assembly of FIG. 18;

FIG. 23 is a schematic illustration of an electronic device in wireless communication with a receiver of the volatile material dispensers disclosed herein;

FIG. 24 is block diagram illustrating various inputs and outputs of the volatile material dispensers disclosed herein; and

FIG. 25 is a flow diagram illustrating an exemplary method of controlling the volatile material dispensers disclosed herein.

### DETAILED DESCRIPTION OF THE DRAWINGS

The present disclosure relates to volatile material dispensing systems and methods that provide for varying a temperature of a heating element to vary one or more fragrance characteristics of a volatile material within a refill to control and enhance user experience and to reduce habituation of a user. The use of pin-point heater technology, i.e., a small heater that reaches a desired temperature very quickly (e.g., milliseconds, seconds, etc.), allows for varying, but targeted amounts of heat from a heater. By varying the heat applied to fragrance, fragrance characteristics of the volatile material can be controlled and manipulated to customize and improve a user experience. The systems and methods disclosed herein allow a user to vary a power input, and thus a temperature of the heating element, to adjust a scent profile to achieve different fragrance characteristics.

By varying the temperature of the heating element in a targeted fashion, a user can modify the fragrance characteristics in such a way that reduces habituation without requiring multiple fragrances or refills. Similarly, the user can select a preferred scent profile to emit from the device that is based upon a specific temperature profile, which allows a user to control their own fragrance experience. The systems and methods disclosed herein address adaptation and scent habituation while providing users with an ability to create a custom fragrance experience through the use of a single refill containing a fragrance or fragrance mixture and are relatively easy and economical to implement compared with systems that require the use of multiple refills. Throughout the disclosure, the terms “about” and “approximately” refer to a range of values ±5% of the numeric value that each term precedes. As noted herein, all ranges disclosed within this application are inclusive of the outer bounds of the range.

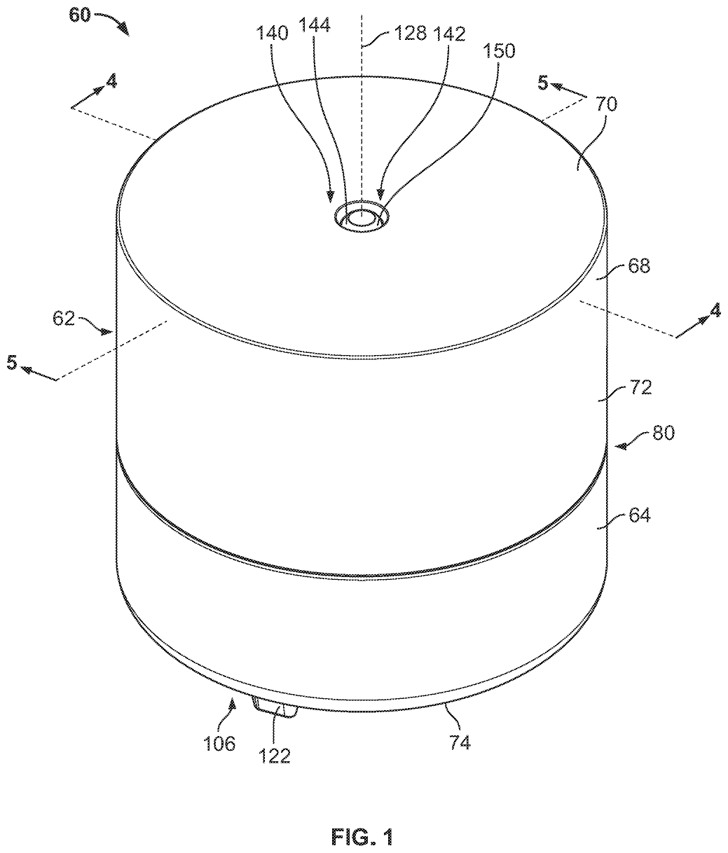

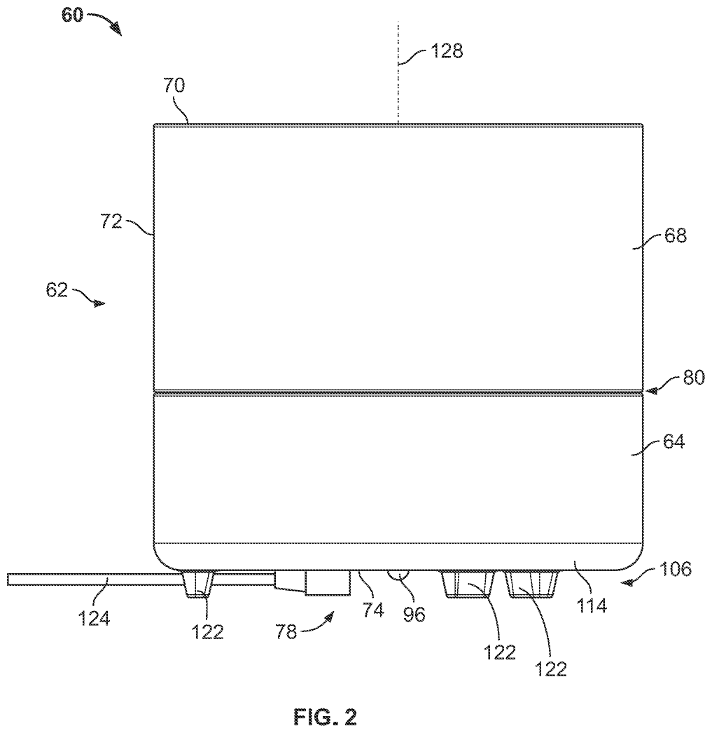

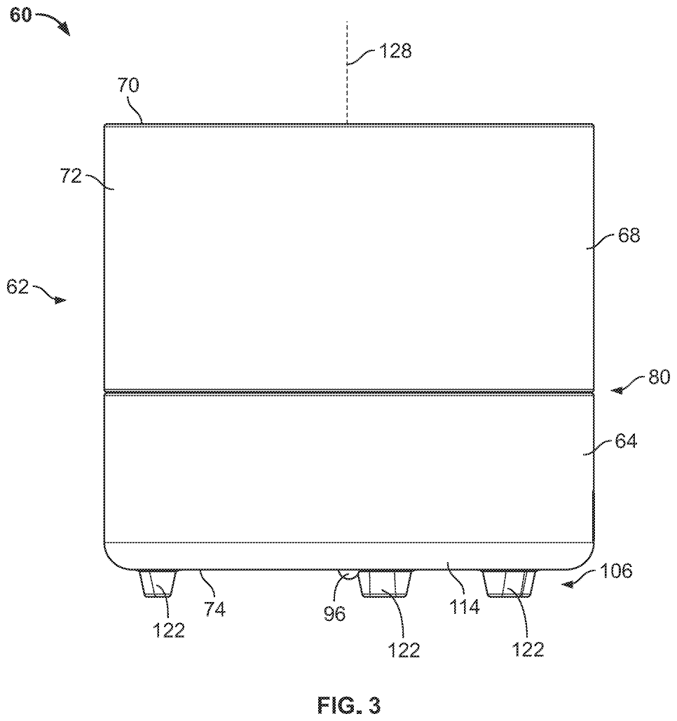

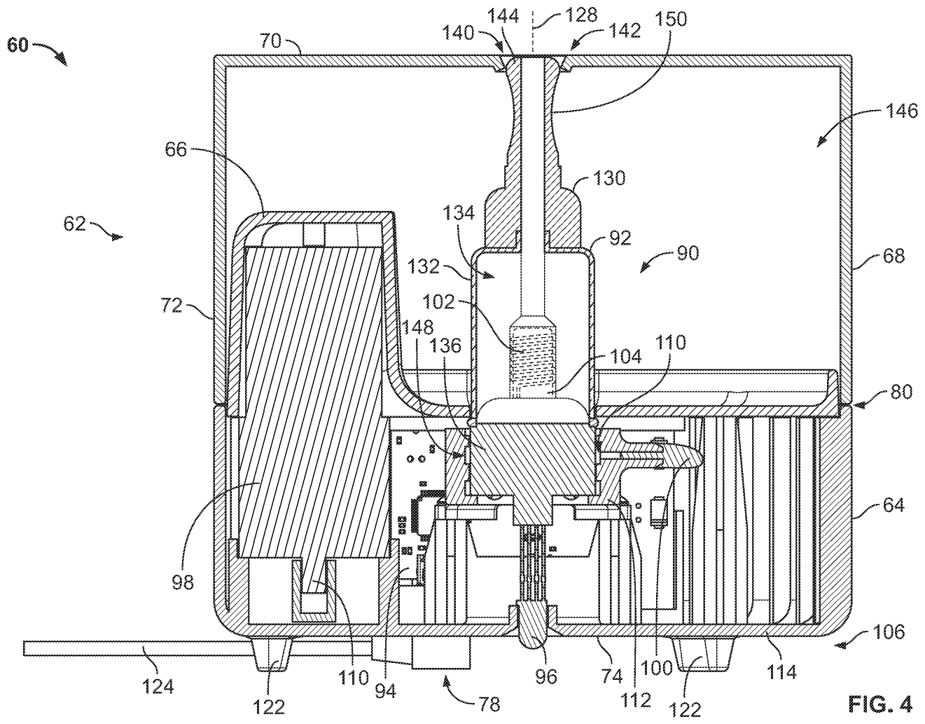

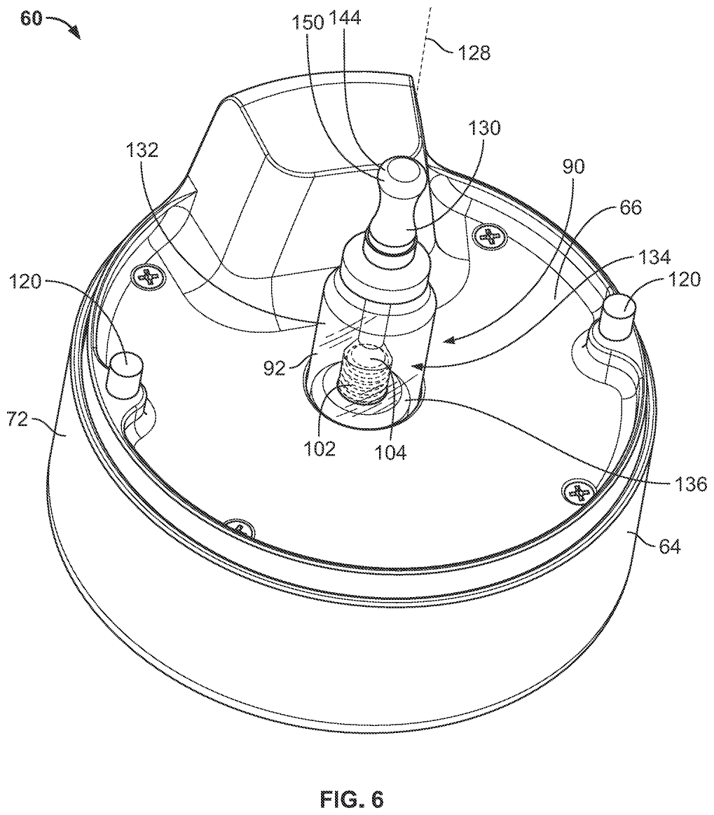

Referring to FIGS. 1-6, a volatile material dispenser 60 is shown, which includes a housing system 62 having a lower housing 64, an upper housing 66 (see FIGS. 4-6), and a cover 68. The housing system 62 further defines an upper surface 70, a sidewall surface 72, and an underside or lower surface 74. The surfaces 70, 72, 74 define outermost surfaces of the housing system 62. Referring in particular to FIGS. 1-3, the volatile material dispenser 60 includes an input port 78, which may receive a plug such as a USB-C type plug, that can be used to charge or power the volatile material dispenser 60. In some embodiments, the volatile material dispenser 60 may be battery operated. The housing system 62 defines a generally cylindrical profile, with the lower housing 64 joining the cover 68 at a seam 80. In some embodiments, the lower housing 64 joins the upper housing 66 at the seam 80, and the upper housing 66 may be removably coupled with the lower housing 64.

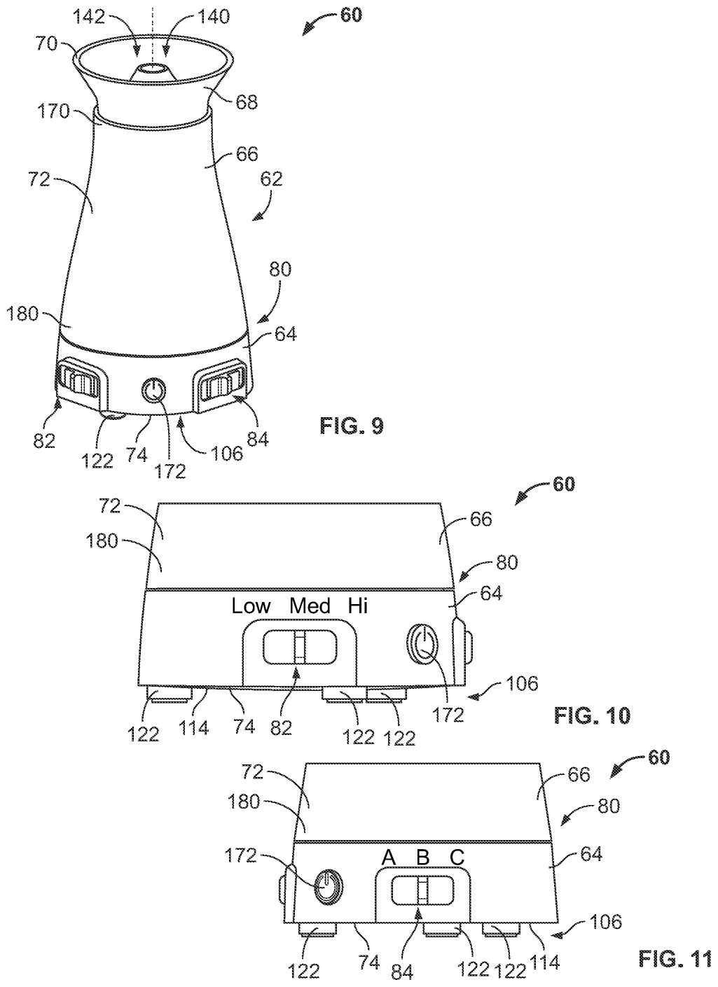

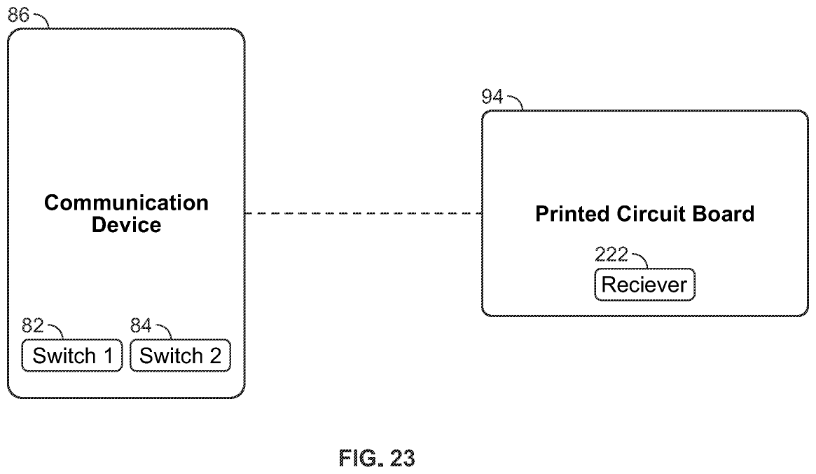

The embodiment of FIGS. 1-6 does not include external switches; however, the volatile material dispenser 60 may include a first switch 82 (see FIGS. 9-11) that allows a user to control an intensity of the fragrance, e.g., low, medium, or high, and a second switch 84 (see FIGS. 9-11) that allows a user to control fragrance character settings, e.g., apple, apple/cinnamon, cinnamon. As illustrated in the block diagram of FIG. 23, the first switch 82 and the second switch 84 may additionally or alternatively be provided via a display screen of an electronic device 86 that is in communication with one or more of the electrical components within the volatile material dispenser 60. It is contemplated that the first switch 82 and the second switch 84 may be provided along one or more of the surfaces 70, 72, 74 of the housing system 62 of FIGS. 1-6, and in particular, along the sidewall surface 72 of the lower housing 64.

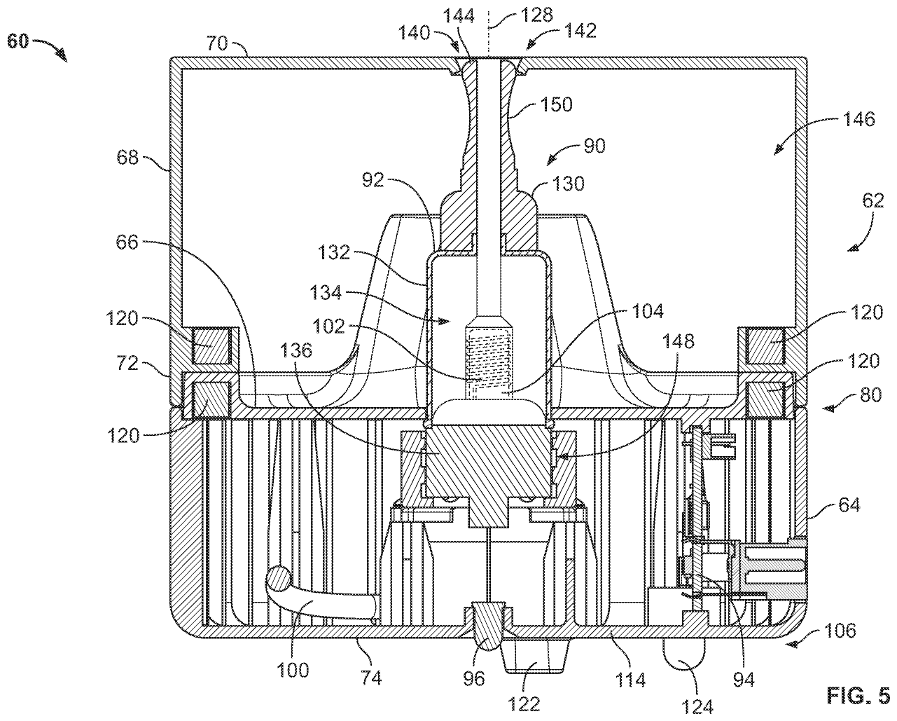

Referring now to FIGS. 4 and 5, a dispensing system 90 of the volatile material dispenser 60 is shown in detail, which includes a refill 92, a control printed circuit board (PCB) 94, at least one light source 96, an air displacement mechanism 98, a hose 100, and a heater 102. A wick 104 is also disposed within the heater 102, the wick 104 being configured to draw fluid into a heating chamber defined by the heater 102. The heater or heating element 102 is configured to heat a volatile material (not shown) within the refill 92. The volatile material may include one or more compositions, which may be any suitable liquid or liquids, and may include one or more active ingredients. Active ingredients include, but are not limited to, one or more of a cleaner, an insecticide, an insect repellant, an insect attractant, a disinfectant, a mold or mildew inhibitor, an antimicrobial, a fragrance comprised of one or more aroma chemicals, a disinfectant, an air purifier, an aromatherapy scent, an antiseptic, an odor eliminator, a positive fragrancing active material, an air-freshener, a deodorizer, a medicinal component, an inhalant (e.g., for relieving a cough or congestion), or the like, and combinations thereof.

While the air displacement mechanism 98 of FIGS. 1-6 is an air pump, alternative air displacement mechanisms 98 are contemplated such as a fan (see FIGS. 9-17), a diaphragm, a vacuum pump, or a compressor. The at least one light source 96 is also shown, which is configured as a light emitting diode (LED) that extends from an underside 74 of the lower housing 64 into a gap 106 that is disposed between a ground engaging surface (not shown) and the underside 74 of the lower housing 64. The air displacement mechanism 98 is disposed between the upper housing 66 and the lower housing 64 along an outer periphery of the lower housing 64. The air displacement mechanism 98 includes a fluid or air channel 110 at a lower end thereof, through which air travels into the hose 100 that connects the air displacement mechanism 98 with a bottom end of the refill 92. The air channel 110 is fluidly coupled with a bottom end of a refill retainer 112, which is centrally located within the dispenser 60. In the present embodiment, the refill retainer 112 is a base or pedestal that extends upward from a lower wall 114 of the lower housing 64; however, the refill retainer 112 may be located along or extend from other portions of the housing structure 62. The heater 102 is further shown as a coil that surrounds the wick 104, the wick being configured to draw liquid into a heating chamber disposed centrally within the refill 92.

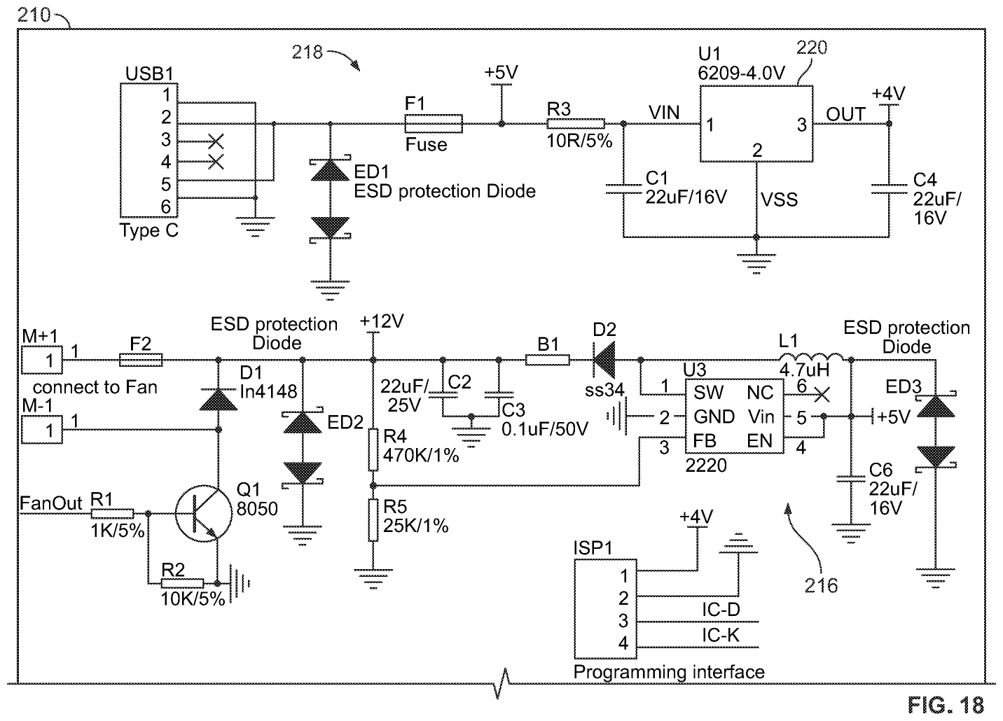

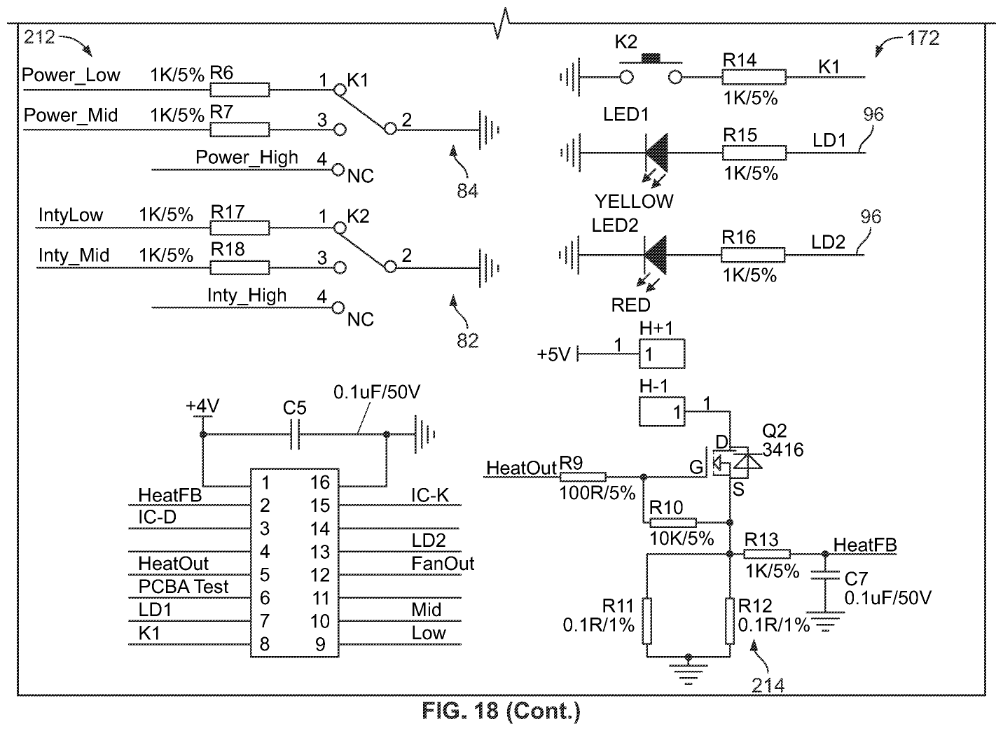

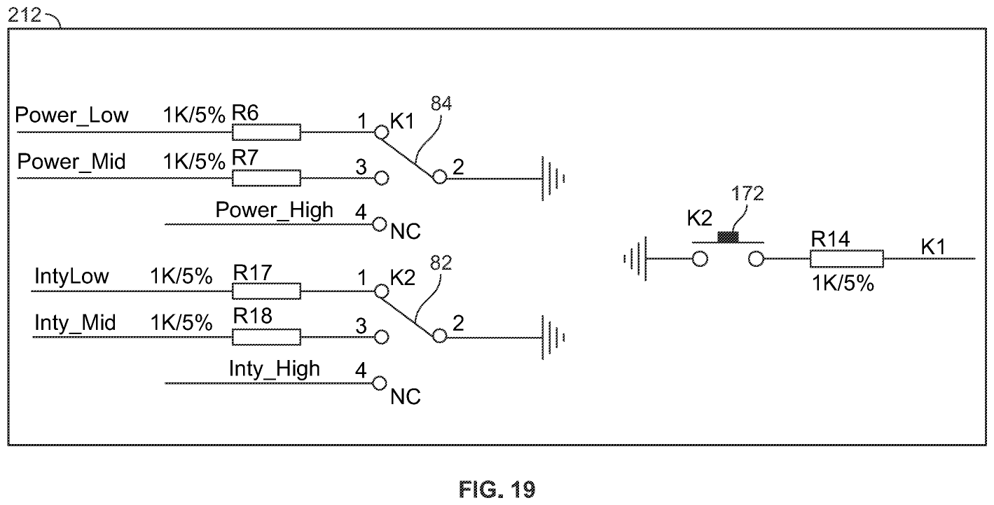

Still referring to FIGS. 4 and 5, The PCB 94 includes various electrical components, which are described in detail with respect to FIGS. 18-24. Referring to FIG. 5, a plurality of magnets 120 are shown, which allow for the cover 68 and the upper housing 66 to be removably coupled to one another. Alternatively, the lower housing 64 and the upper housing 66 may be permanently secured to one another, e.g., via fasteners, such as screws, an adhesive, ultrasonic welding, etc. The light source 96 is disposed centrally along the underside 74 of the housing system 62. A plurality of feet 122 depend downward from the lower housing 64 and define the gap 106 when the volatile material dispenser 60 is placed on the resting surface. An external power cord 124 extends from the underside 74 of the housing system 62, the external power cord 124 being removably coupled with the input port 78, e.g., a USB-C socket, of the volatile material dispenser 60.

The refill 92 is shown in detail in FIGS. 4-6 and is disposed centrally within the volatile material dispenser 60. A longitudinal axis 128 is defined through a center of the refill 92. In the present embodiment, the longitudinal axis 128 defines a center of both the refill 92 and the housing system 62. However, in alternative embodiments, the refill 92 may define the longitudinal axis 128, and the housing system 62 may define a different longitudinal axis and may take varying forms. In some embodiments, and as shown in FIGS. 4-6, the refill 92 includes a cartridge 130, and may include additional elements, such as a base (not shown) that is coupled with the cartridge 130. In some embodiments, the cartridge 130 may be a glassomizer, a clearomizer, or a catromizer, or may be another type of device that is used to deliver liquid to vapor. In the illustrated embodiment, the cartridge 130 includes a tank 132 defining a chamber 134. A refill base 136 can be coupled with the cartridge 130 to define the refill 92. The refill base 136 may be configured as a coupling member that allows the refill 92 to be coupled to one or more components within the system 60. The heater 102 as described above is configured to heat up a liquid (not shown) that is disposed within the chamber 134 of the tank 132. As an example, the cartridge 130 may be an Aspire™ Nautilus tank that implements an adjustable airflow ring at a bottom end thereof that allows for various heat settings. The cartridge 130 includes the removable tank 132, which may comprise Pyrex®.

Referring to FIGS. 1, 4, and 5, the upper housing 66 further includes an upper aperture 140 that provides an outlet 142 through which a volatile material is dispensed. The outlet 142 is in direct fluid communication with at least a nozzle end 144 of the refill 92 and may further be in communication with an interior cavity 146 that is defined between the cover 68 and the upper housing 66. In some embodiments, the interior cavity 146 is defined between the upper housing 66 and the lower housing 64. However, in the illustrated embodiment, the interior cavity 146 is not in direct fluid communication with the upper aperture 140, due to the functionality of the air dispensing system 90, i.e., the air displacement mechanism 98 pushes air through the hose 100, which enters an emission cavity 148 within the refill retainer 112, mixes with the volatized material within the tank 132 of the refill 92, and is expelled out of a nozzle 150 of the refill 92 due to a pressure differential caused by the air displacement mechanism 98. In other embodiments, such as the embodiment described below, the air displacement mechanism 98 may be a fan that expels air around and/or through the chamber 134 of the refill 92, causing the volatile material to be expelled from the housing system 62.

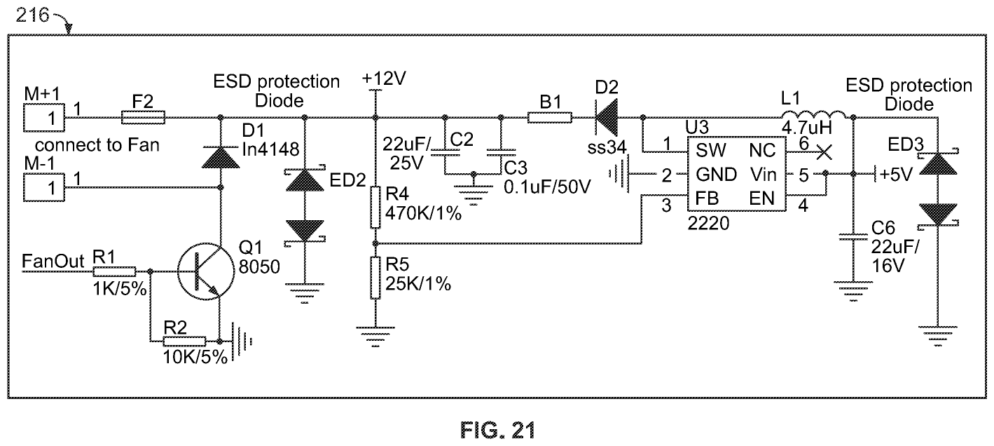

As discussed further below, the volatile material dispenser 60 also includes features that provide enhanced customization and adaptation capabilities. Algorithms are used to modify dispenser operation parameters according to user preferences and/or volatile material requirements. Generally, a controller or controller stage (see FIG. 21) operates the dispenser 60 according to pre-programmed sequences, which are designed to mitigate habituation. More specifically, algorithms are used to vary the temperature applied to the heater, as well as the output of the dispensing system 90, e.g., controlling the speed or revolutions per minute (RPM) of the air displacement mechanism 98. For example, pulse width modulation (“PWM”) may be used to adjust the heater 102 to different temperatures according to various duty cycles. Varying the temperature of the heater 102 and speed of the fan results in varied fragrance intensities, which prevents a user from acclimating to a fragrance. The operation of the dispenser 60 may be adjusted directly on the dispenser 60 through user manipulated controls or wirelessly through a user's mobile device (see FIG. 23). In some embodiments, the heater 102 is configured to achieve a temperature of between about 125° F. and about 275° F., or between about 150° F. and about 250° F., or between about 175° F. and about 225° F. The heater 102 may comprise one or more resistors having a resistance of between about 0.5 Ohms (Ω) and about 15.0Ω, or between about 1.5Ω and about 10.0Ω, or between about 2.5Ω and about 7.5Ω, or between about 3.5Ω and about 5.0Ω.

The present disclosure relates to volatile material dispensers that are configured to emit any fragrance within any fragrance or combinations of fragrances. More specifically, the volatiles configured for use with the volatile material dispensers 60 disclosed herein may include any of the perfume raw materials that are listed within U.S. Pat. No. 9,855,361, which is incorporated by reference herein in its entirety. As discussed above, many fragrances comprise a number of different perfume raw materials, each having their own chemical characteristics or properties, which generally vary in terms of polarity, density, vapor pressure, flash point, and other properties. By identifying the chemical characteristic of vapor pressure of various perfume raw materials or combinations of perfume raw materials, a targeted fragrance character or characteristic can be released from the volatile material dispenser 60. By referencing the vapor pressure of an identified fragrance, a particular amount of power or heat may be applied to the volatile within the refill 92, which can achieve a targeted fragrance characteristic from within the volatile, allowing for the reduction of scent habituation.

The term “fragrance,” as used herein, refers to any substance or a mixture of substances such as a perfume designed to emit an aromatic scent. A wide variety of chemicals are known for fragrance (i.e., perfume) uses, including materials such as aldehydes, ketones, and esters. More commonly, naturally occurring plant and animal oils and exudates comprising complex mixtures of various chemical components are known for use as fragrances.

In some embodiments, the fragrances of the present application may comprise a single chemical or may comprise a sophisticated complex mixture of natural and synthetic chemical components, all chosen to provide any desired odor. For example, the fragrances of the present application may comprise one or more perfume raw materials. The term “perfume raw materials,” as used herein, refers to any compound (e.g., those having molecular weight of at least 100 g/mol) or substance that are useful in imparting an odor, fragrance, essence, or scent either alone or in combination with other “fragrance raw materials.” Mixtures of fragrance raw materials are known by those skilled in the art of fragrances and perfumes as “accords.” The term “accord,” as used herein, refers to a mixture of two or more fragrance raw materials which are artfully combined to impart a scent, odor, essence, or fragrance characteristic.

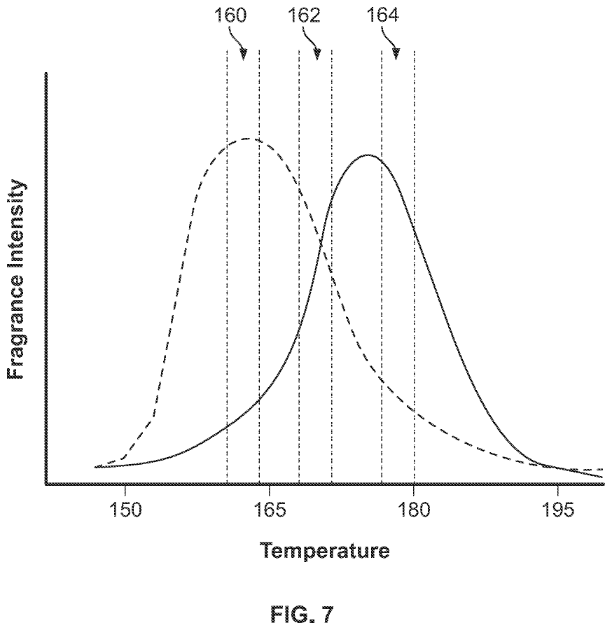

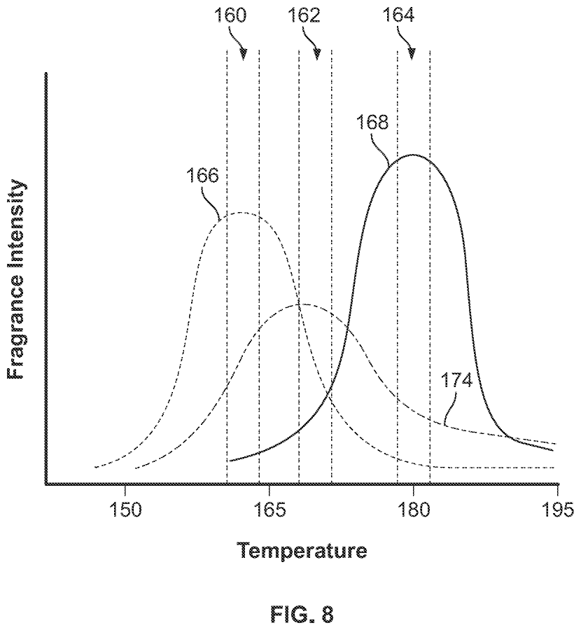

Referring now to FIGS. 7 and 8, two graphs are provided that illustrate varying fragrance characteristics comparing temperature and fragrance intensity. Referring to FIG. 7, a bi-modal graph is provided, which depicts an apple-cinnamon accord that is released based on a power input or temperature of the heater 102. In the particular example of FIG. 7, less power or heat is required to emit a first fragrance defining a first fragrance characteristic 160 that smells more like “apple” (e.g., a first power or first temperature), somewhat more heat is required to cause the volatile material dispenser 60 to emit a second fragrance defining a second fragrance characteristic 162 that smells more like “apple-cinnamon” (e.g., a second power or second temperature), and even more heat is required to cause the volatile material dispenser 60 to emit a third fragrance defining a third fragrance characteristic 164 that smells more like “cinnamon” (e.g., a third power or third temperature). The first fragrance characteristic 160 that defines an “apple” scent is the result of an optimum temperature being achieved for a first composition 166, and the third fragrance characteristic 164 that defines a “cinnamon” scent is the result of an optimum temperature being achieved for a second composition 168. A third composition 170 may also be included, as discussed in detail below.

Referring to FIG. 8, a tri-modal accord is illustrated, which may comprise “lavender,” “jasmine,” and “vanilla” fragrance characteristics, for example. In some embodiments, one or more additional fragrance characteristics may be included, and each fragrance characteristic may have a different optimal fragrance release temperature, as shown by the peaks within the graphs of FIGS. 7 and 8. In the example of FIG. 8, the first fragrance characteristic 160 smells more like “lavender” with a hint of “jasmine” and “vanilla,” the second fragrance characteristic 162 smells more like “jasmine” with more subtle notes of “lavender” and “vanilla,” and the third fragrance characteristic 164 smells more like “vanilla” with hints of “jasmine” and “lavender.” More fragrance characteristics may be included, and additional, non-fragranced compositions may be included and configured for release within one or more temperature schemes, as described in greater detail below.

By switching the heater 102, via the second switch 84, to a lower or first setting, the user can emit the first fragrance characteristic 160, by switching the heater 102 to the middle or second setting, the user can emit the second fragrance characteristic 162, and by switching the heater 102 to a higher or third setting, the user can emit a third fragrance characteristic 164, based upon the heating characteristics of the compositions 166, 168. Referring again to the graph of FIG. 7, the first fragrance characteristic 160 corresponds with an “apple” fragrance with a hint of “cinnamon,” the second fragrance characteristic 162 corresponds with a relatively equal mixture of “apple” and “cinnamon,” and the third fragrance characteristic 164 corresponds with “cinnamon” with a hint of “apple.” By allowing for three different fragrance characteristics to be emitted from the dispenser 60 based on a selected input from a user, the dispenser 60 is configured to provide a user the ability to select, within pre-determined minimum and maximum limits, how a fragrance will smell.

It should be noted that intensity selection is also capable via the first switch 82 of the dispenser 60, e.g., an intensity selection of high, medium, or low. The first switch 82 controls an amount of time of activation and/or an amount of time between activations or operation cycles. The fragrance characteristic selection allows an end user to be able to select a fragrance characteristic by manipulating the second switch 84 between two or more fragrance characteristic selections. When incorporated along the dispenser 60, the first switch 82 and the second switch 84 may comprise one or more of a multi position selector switch, capacitive switch, or another type of switch. In the illustrated embodiments of FIGS. 7 and 8, the first, second, and third fragrance characteristics 160, 162, 164 are associated with different, pre-determined wattages, which can be pre-determined based on an identified vapor pressure of each of the particular fragrances to be emanated from the dispenser 60 that corresponds with a temperature that is achieved based upon a specified power setting, e.g., 5.0 W, 5.5 W, and 6.0 W. In some embodiments, the second switch 84 may include discrete positions such that only a pre-determined number of fragrance characteristics may be implemented, e.g., 2, 3, 4, etc. However, in alternative embodiments, an infinite number of fragrance characteristics may be implemented. To that end, the second switch 84 may be a dial (not shown) that is movable between a minimum setting and a maximum setting and may be adjustable to achieve an infinite number of temperature settings. Although ranges of 5.0 to 6.0 W are discussed, various other heating profiles are possible (e.g., between 1.0 W and 9.0 W), and can be chosen based upon the fragrance present within the refill 92.

In some embodiments, the first fragrance characteristic 160 is achieved using a temperature scheme among one of the following temperature schemes, although varying power schemes may also be utilized depending on the type of fragrance to be emitted. In some embodiments, the first fragrance characteristic 160 is achieved at a temperature within a first range of between about 150° F. and about 200° F., or between about 160° F. and about 190° F., or about 175° F. In some embodiments, the second characteristic is achieved at a temperature within a second range of between about 175° F. and about 225° F., or between about 185° F. and about 215° F., or about 200° F. In some embodiments, the third fragrance characteristic is achieved at a temperature within a third range of between about 200° F. and about 250° F., or between about 210° F. and about 240° F., or about 225° F. In some embodiments, a fragrance profile may be identified to align with a pre-defined temperature scheme of the dispenser 60, e.g., 175° F., 200° F., and 225° F. To that end, a first fragrance may be identified that has a fragrance characteristic that is achieved within the first temperature range, a second fragrance may be identified that has a fragrance characteristic that is achieved within the second temperature range, and a third fragrance may be identified that has a fragrance characteristic that is achieved within the third temperature range.

With respect to FIG. 8, the second switch 84 allows a user to choose the optimum temperature that allows for the fragrance characteristics to be achieved that are associated with the first composition 166, the second composition 168, and the third composition 170. The first composition 166 defines the first fragrance characteristic 160 (e.g., “lavender”), the second composition 168 defines the second fragrance characteristic 162 (e.g., “jasmine”), and the third composition 170 defines the third fragrance characteristic 164 (e.g., “vanilla”). The fragrance characteristics 160, 162, 164 may be associated with a peak in fragrance intensity of the chemical compositions 166, 168, 170, or the fragrance characteristics may be associated with relative increases in fragrance intensities.

To that end, the third fragrance characteristic 164 is shown associated with an elevated fragrance intensity of the third composition 170 but may not be associated with the peak intensity. In comparison, the first fragrance characteristic 160 is associated with a peak fragrance intensity of the first composition 166 in the example of FIG. 8. In some embodiments, the fragrance characteristics are associated with peak fragrance intensities of the respective compositions, or only some of the fragrance characteristics are associated with peak fragrance intensities. Still further, in some embodiments, the fragrance characteristic may be associated with a non-fragranced active, such as a pest control active, which may be volatized at a higher rate than other compositions within the refill 92. In such an embodiment, the fragrance characteristics are reflective of an attribute or characteristic of the composition that is non-fragrance based.

Referring again to FIG. 7, in some embodiments the first composition 166 is a non-fragranced pest control active and the second composition 168 is a fragrance. In such an embodiment, the non-fragranced pest control product may be emitted at a higher concentration, at a lower temperature, or vice versa, and the fragrance may be emitted at a higher concentration, at a higher temperature, or vice versa. Since the second composition 168 containing the fragrance is not as perceptible at a first or lower temperature but is perceptible at a second or higher temperature that is different than the first temperature, the dispenser 60 disclosed herein can be used as a pest control product that has the ability to operate in both an unfragranced and a fragranced repellent mode.

In some embodiments, a fourth switch (not shown) may be implemented, which may be configured to allow a user to manually choose a cartridge setting based on a type of cartridge 130 or a fragrance or volatile material within the cartridge 130. In some embodiments, the fourth switch may be adjustable between 2, 3, 4, 5, 6, 7 or more cartridge settings, and each of the cartridge settings may be associated with a particular or pre-identified power scheme. As a result, a first cartridge may be associated with a first power scheme, a second cartridge may be associated with a second power scheme different than the first power scheme, and a third cartridge may be associated with a third power scheme different than the first and second power schemes. In some embodiments, the first cartridge may be associated with a first fragrance, the second cartridge may be associated with a second fragrance different than the first fragrance, and the third cartridge may be associated with a third fragrance different than the first and second fragrances. The fourth switch (not shown) may be a slider switch or may be another type of switch as disclosed herein. Further, the fourth switch may be accessible from the display screen of the electronic device 86.

In some embodiments, the fragrance within the cartridge may be identified based on one or more cartridge characteristics that are manually input by a user, or automatically detected via one or more sensors within the dispenser 60. To that end, the dispenser 60 may include one or more sensors, such as an infrared (IR) sensor, an optical sensor, a weight sensor, a hall effect sensor, one or more magnets, a radio frequency identification (RFID) sensor, a barcode scanner, a QR scanner, a proximity sensor, a reflective photo interpreter, a humidity sensor, a fluid property sensor, a light sensor, an alcohol sensor, or another type of sensor. In some embodiments, the electronic device 86 may be configured to retrieve data that provides information to the dispenser 60 regarding the collected sensory input from the above-mentioned sensors. For example, the electronic device 86 may be configured as a QR scanner, e.g., via a camera, that may scan a QR code along an exterior of the cartridge 130.

In some embodiments, data from one or more of the above-referenced sensors (not shown) may be received by a memory and/or a processor, as described in greater detail below, and information stored within a lookup table may be compared against data that is retrieved by any of the sensors. An identified fragrance or fragrance characteristic of the volatile material may be determined based on the comparison of the data with the information stored within the lookup table. However, as noted above, a user may manually input information, e.g., via the fourth switch, that identifies a cartridge type. In some embodiments, the cartridge includes two fragrances that are configured to release two different fragrance characteristics, i.e., the cartridge is a 2-tone cartridge (as depicted in FIG. 7). In some embodiments, the cartridge includes three fragrances that are configured to release three different fragrance characteristics, i.e., a 3-tone cartridge (as depicted in FIG. 8). In some embodiments, the cartridge is configured to release four different fragrance characteristics, i.e., a 4-tone cartridge. In some embodiments, a volatile material may be identified based on data that is retrieved by the one or more sensors noted above in combination with data manually input by a user.

In some embodiments, a lock-and-key feature is provided with the cartridge 130, e.g., a protrusion along an upper rim of the cartridge 130, which may provide information to the dispenser 60 regarding the volatile material within the cartridge 130. The lock-and-key feature may be disposed along the exterior surface of the cartridge 130 or may be provided along an inner surface of the cartridge 130, e.g., along a channel defining a mouth of the cartridge 130. The dispenser 60 may be configured to detect a type of volatile material that is within the cartridge 130 based on a location of the lock-and-key feature, e.g., an apple-cinnamon cartridge may be identified based on a location of two radially offset protrusions along an exterior surface of the cartridge 130.

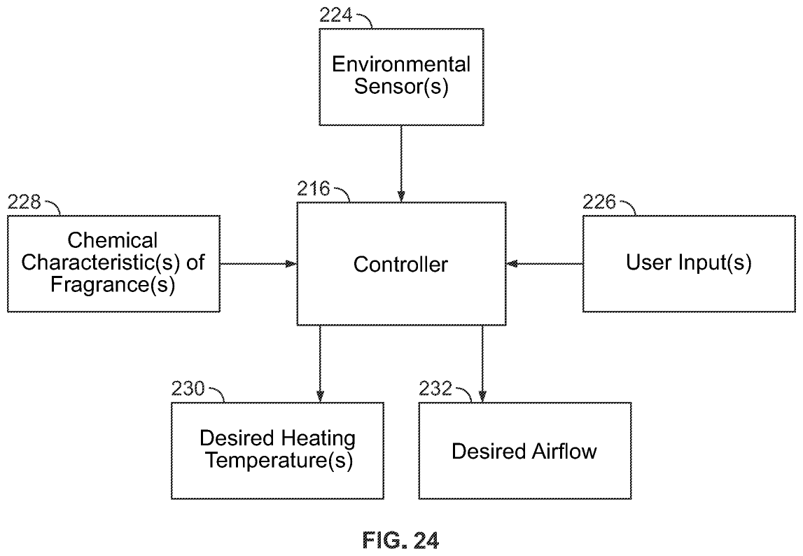

In some embodiments, the power limits may be variable or different for different fragrances or desired fragrance characteristics. Further, the intensity limits, e.g., on time/off time between activations, may also be different among different fragrances. In some embodiments, both fragrance character and intensity limits may be changed throughout operation of the volatile material dispenser 60, e.g., due to changes that may occur over time or due to one or more of a number of external factors that may be measured or identified either automatically or from information input by a user. In some embodiments, automatic triggers may cause the fragrance character or intensity limits, e.g., minimum and maximum switch positions, to be adjusted, for example based on a time of the day, a measured room temperature, a season of the year, or vital signs of a person acquired from the electronic device 86 (see FIG. 23), e.g., a cell phone, a smart watch, or another type of device having sensors or that can receive user inputs. The user inputs may be received from environmental sensors (see FIG. 24), which may include external sensors along any number of devices that are configured to communicate either directly with the volatile material dispenser 60, or with the electronic device 84, and that are configured to provide feedback to the dispenser 60. The changes in intensity levels can also be triggered via request from an app or via direct physical interaction with the dispenser 60.

The different fragrance characteristics are emitted when the refill 92 is in thermal communication with the heater 102, which may be a pin-point heater. By using a heater such as the heater 102 disclosed herein, desirable temperatures can be achieved relatively quickly. In some embodiments, the heater 102 may be turned “on” for few seconds, every few minutes. During the activation period, the power level delivered to the heater 102 can be varied (e.g., between a range of power settings) to achieve an average target power setting (e.g., 5.0 W) over the activation period. In other examples, the heater 102 is activated and held at a constant power level throughout the activation. Through the use of PWM, the heater 102 can be adjusted to a range of different temperatures, and by achieving varying temperatures, different fragrance characteristics can be produced and output. Since small deviations in temperature may not cause a different fragrance characteristic that is perceptible by a user, some embodiments of the present disclosure implement discrete settings that provide for different fragrance characteristics that are perceptible by a user, e.g., “apple,” “apple-cinnamon,” and “cinnamon.”

In some embodiments, the fragrance characteristics can be enhanced through different durations of operation, different frequencies of operation, e.g., every 5 minutes vs. every 15 minutes, an algorithm that varies the temperature of activation in a predetermined fashion, an on/off timer, or varying a speed of the air displacement mechanism 98 when the heater 102 is turned “on.” In the embodiment described above with respect to FIGS. 1-6, a user is capable of controlling the various parameters discussed above through an app, which allows a user to vary characteristics of the fragrance by choosing different pre-determined operational points that are connected to fragrance characteristics. In some embodiments, the operational points may be pre-determined or pre-programmed. While the embodiments disclosed herein are focused on pin-point heaters, plug-in scented oil (PISO) heaters could also be used with the volatile material dispensers disclosed herein.

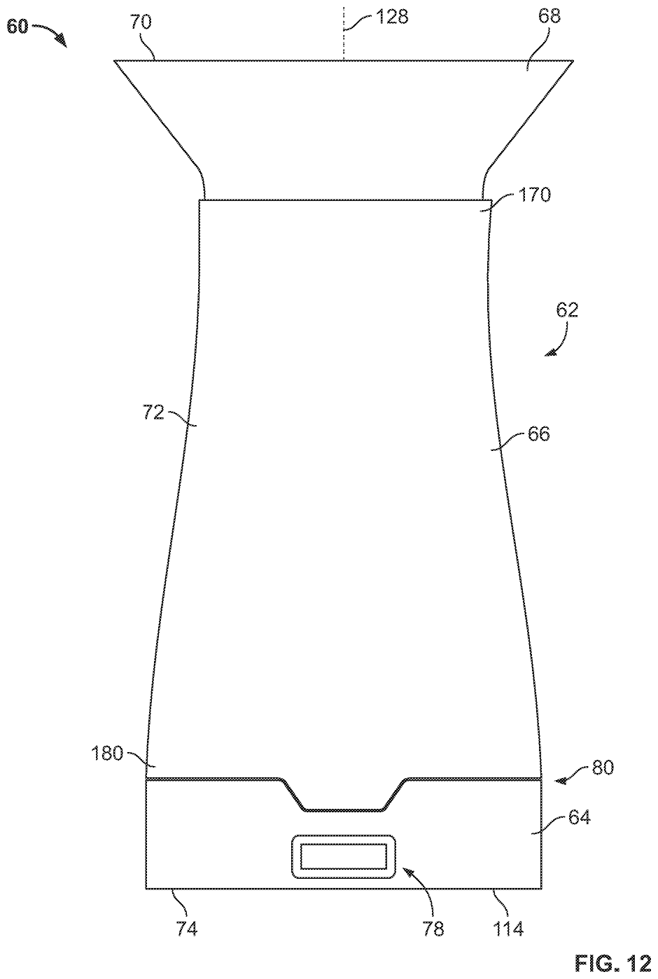

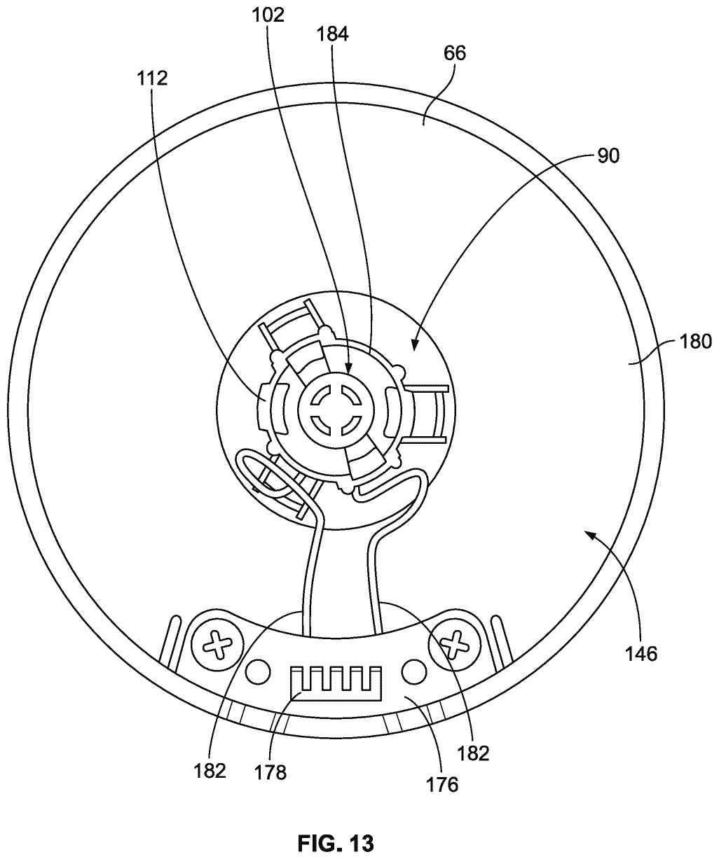

Referring now to FIGS. 9-17, another embodiment of the volatile material dispenser 60 is shown. The volatile material dispenser 60 includes the housing system 62, including the lower housing 64, the upper housing 66, and the cover 68. A third switch 174 is shown along the lower housing 64, which is described in greater detail below. In the present embodiment, the upper housing 66 is externally visible, and the cover 68 is outwardly flared from an upper end 172 of the upper housing 66. Referring to FIG. 10, the first switch 82 is shown, which allows a user to control an intensity of the fragrance, e.g., low, medium, or high. Referring to FIG. 11, the second switch 84 is shown, which allows a user to control fragrance character settings, e.g., “apple” (A), “apple/cinnamon” (B), and “cinnamon” (C). Referring to FIGS. 9-11, the third switch 174, which is an external on/off or power switch, is shown, as well as the feet 122 of the volatile material dispenser 60, which define the gap 106 between the underside 74 of the lower housing 64 and the resting surface (not shown). In the present embodiment, the gap 106 allows for air to be drawn into the lower housing 64 via the air displacement mechanism 98, which is a fan in the present embodiment. Referring to FIG. 12, the volatile material dispenser 60 includes the input port 78, which may receive a plug such as a USB-C type plug, which can be used to charge or power the dispenser 60. As noted above, in some embodiments, the volatile material dispenser 60 may be battery operated.

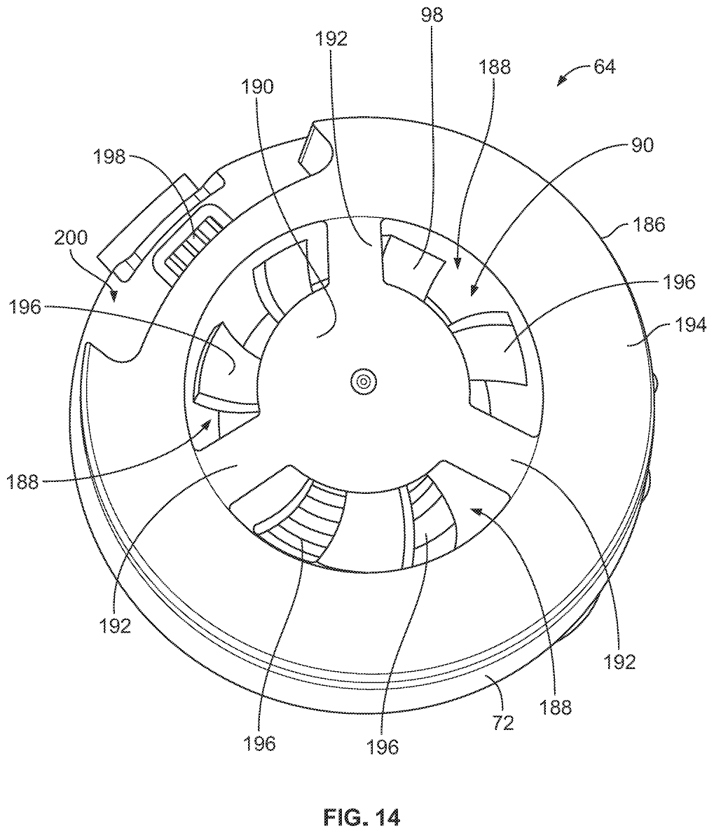

Referring now to FIGS. 13 and 14, various electrical components of the volatile material dispenser 60 are shown. Referring in particular to FIG. 13, a first PCB 176 from which a first terminal 178 extends is shown disposed at a lower end 180 of the upper housing 66, and first and second wires 182 extend from the first PCB 176 toward the upper end 172 of the upper housing 66. The refill retainer 112 is shown disposed at the upper end 172 of the upper housing 66. A plurality of the heaters 102 are disposed along an inner circumferential surface 184 of the refill retainer 112, the heaters 102 being electrically coupled with the PCB 94 and thus the first terminal 178 via the wires 182.

Referring to FIG. 14, an upper end 186 of the lower housing 64 is shown, which includes radially spaced apertures 188. An inner platform 190 is disposed radially inward of the apertures 188, the inner platform 190 being integral with struts 192 that extend radially outward from the inner platform 190 and are integral with an outer platform 194. Fan blades 196 of a fan (air displacement mechanism 98) are visible through the apertures 188. A second terminal 198 is also shown disposed within an inset region 200 of the lower housing 64, the second terminal 198 being electrically coupled with a second PCB (not shown), which is disposed within the lower housing 64 and may be the control PCB 94. Various other electrical components as disclosed herein, such as those discussed in FIGS. 18-24, may be provided along the second PCB. While not depicted in the present embodiment, a plurality of LEDs 96 may be disposed within the lower housing 64, or along the outer surfaces 70, 72, 74 of the housing system 62.

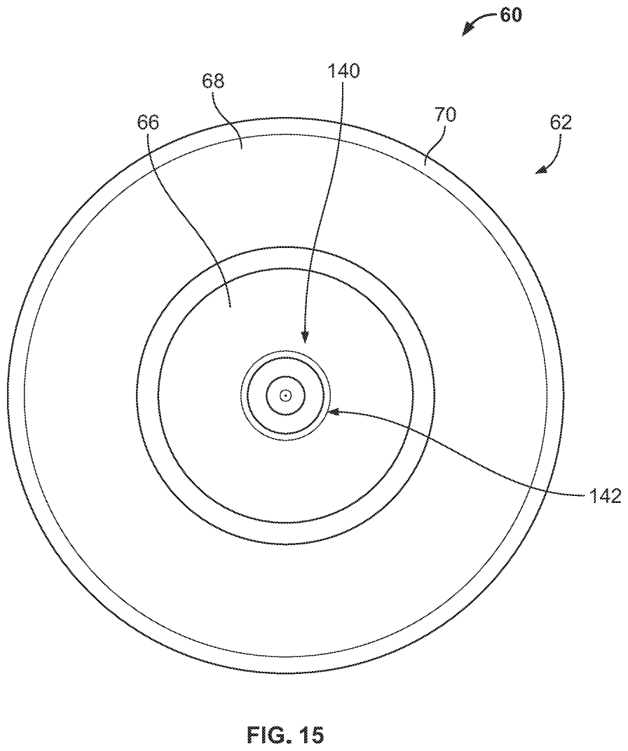

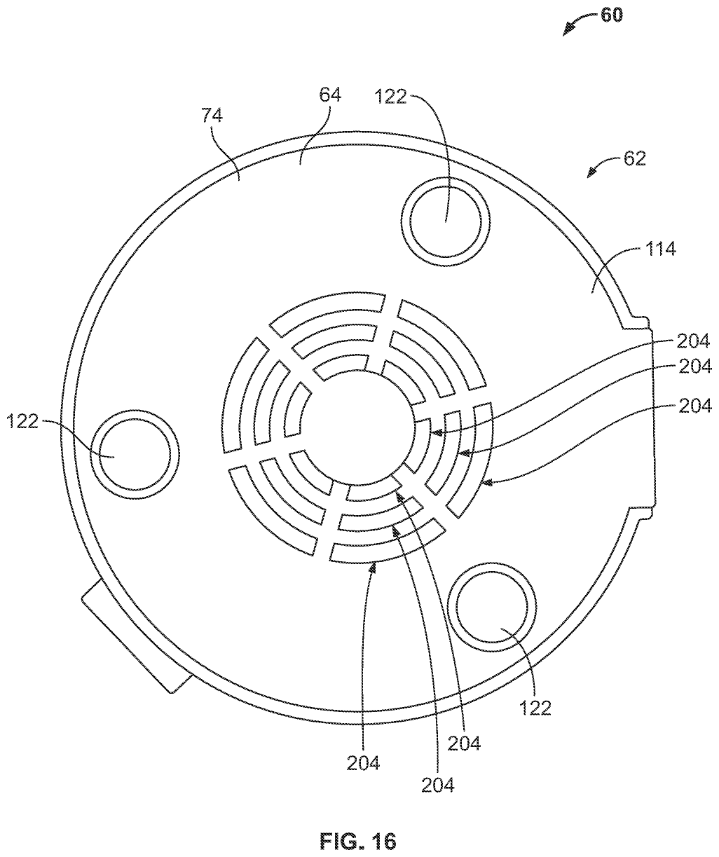

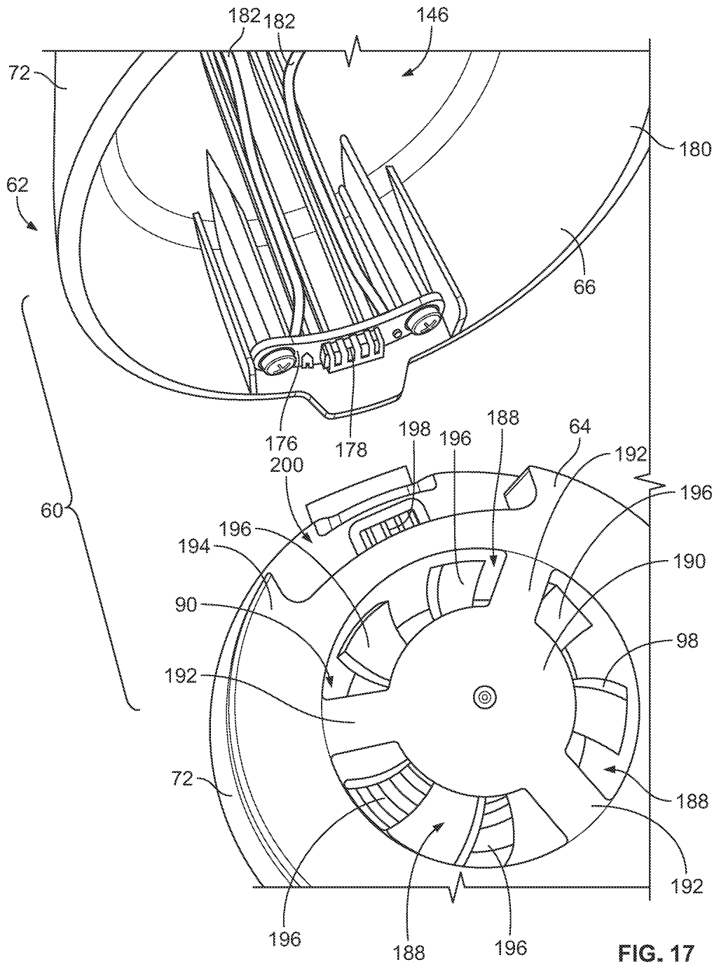

Referring now to FIGS. 15 and 16, top and bottom views of the dispenser of FIG. 9 are shown. Referring to FIG. 15, the cover 68 and the upper housing 66 are shown, as well as the upper aperture 140 through which the volatile is configured to be dispensed. The longitudinal axis 128 extends centrally through the upper aperture 140, and the housing system 62 is generally radially symmetric about the longitudinal axis 128. Referring to FIG. 16, a bottom end of the lower housing 64 is shown, along with a plurality of the feet 122. A plurality of inlet air gaps 204 are provided along the underside 74, which allow air to be drawn into the lower housing 64. As noted above, the lower housing 64 may house a variety of electrical components, as discussed hereinafter below. Referring to FIG. 17, the upper housing 66 and the lower housing 64 are shown adjacent to one another, such that the first terminal 178 is configured to be electrically coupled with the second terminal 198.

Referring now to FIGS. 18-25, various electrical schematics and flow diagrams are illustrated. Referring specifically to FIG. 18, a schematic diagram is shown illustrating an electrical assembly 210 for use in the volatile material dispensers 60 disclosed herein. The electrical assembly 210 includes a user input stage 212, a heating stage 214, a controller stage 216, and a power stage 218. In particular, the power stage 218 is configured to receive power from a power source, such as a wall outlet, a battery, a car lighter socket, or another power source. The power source could be either alternating current or direct current. The power stage 218 manages the received power to operate various electrical components of the electrical assembly 210, including energizing the heating element 102 and the air displacement mechanism 98.

Referring to FIG. 19, the user input stage 212 is shown, which includes the first switch 82, the second switch 84, and the third switch 174. In the embodiment of FIGS. 9-17, the first switch 82 is a three-position slide switch and is configured to control an intensity of the fragrance of the volatile material, the second switch is also a three-position slide switch and is configured to control a character of the fragrance of the volatile material, and the third switch 174 is a push button on/off switch that is configured to control power to the volatile material dispenser 60 from the power source. There may be more or fewer switches that may control other aspects of the electronic assembly 210. In some embodiments, the switches may have 2, 3, 4, 5, 6, 7, 8, or more discrete settings from a minimum limit to a maximum limit. In some embodiments, the switches 82, 84 may have an infinite number of settings from a minimum limit to a maximum limit.

With respect to the first switch 82, which is configured to control an intensity of the fragrance, a first switch setting may define a “low” setting, which may set the heating element 102 to operate with 5 minutes between each operational cycle. A second switch setting may define a “medium” setting that may set the heating element 102 to operate with 3 minutes between each operational cycle. A third switch setting may define a “high” setting that may set the heating element to operate with 1 minute between each operational cycle. By adjusting the first switch 82 between the three or more switch settings, the timing between operational cycles differs so different fragrance intensities can be achieved.

With respect to the second switch 84, which is configured to control a characteristic of the fragrance, a first switch setting may define the first fragrance characteristic 160, which may be “apple,” a second switch setting may define the second fragrance characteristic 162, which may be “apple-cinnamon,” and a third switch setting may define the third fragrance characteristic 164, which may be “cinnamon.” The first fragrance characteristic 160 of “apple” may be associated with 4.0 Watts (W) of power that is delivered to the heating element 102 or the dispenser 60. The second fragrance characteristic of “apple-cinnamon” may be associated with 5.0 W of power that is delivered to the heating element 102 or the dispenser 60. The third fragrance characteristic 164 of “cinnamon” may be associated with 6.0 W of power that is delivered to the heating element 102 or the dispenser 60. Other electronic components, such as the air displacement mechanism 98 may draw power, so less power may be delivered to the heater 102 than is delivered to the entire system. In some embodiments, 4.0 W of power may be associated with a “low” setting, 5.0 W of power may be associated with a “medium” setting, and 6.0 W of power may be associated with a “high” setting. As noted above, the different fragrance profiles may be defined by any two fragrance profiles defining different vapor pressures. In a preferred embodiment, the different fragrance profiles are perceptible by a user so as to reduce habituation.

The third switch 174 is configured as a power switch and is used to power “on” and “off” the volatile material dispenser 60. When the third switch 174 is activated to an “on” position, the controller stage 216 can direct electric current to flow to the heating element 102 and the air displacement mechanism 98, using power supplied by the power stage 218. Once a target temperature associated with the position of the second switch 84 is achieved, the controller stage 216 can cut off the electrical current supplied to the heating stage 214 while continuously monitoring a current temperature of the heating element 102 using signals provided by a thermistor or another environmental sensor arranged on or in proximity to the heating element 102. When the measured temperature drops to a predetermined value, the controller stage 216 may restore electrical current to initiate another operation cycle. In some embodiments, a pulse width modulation (PWM) algorithm may be used to allow the heating element 102 to heat up quickly, which in turn would allow a faster fragrance or volatile release.

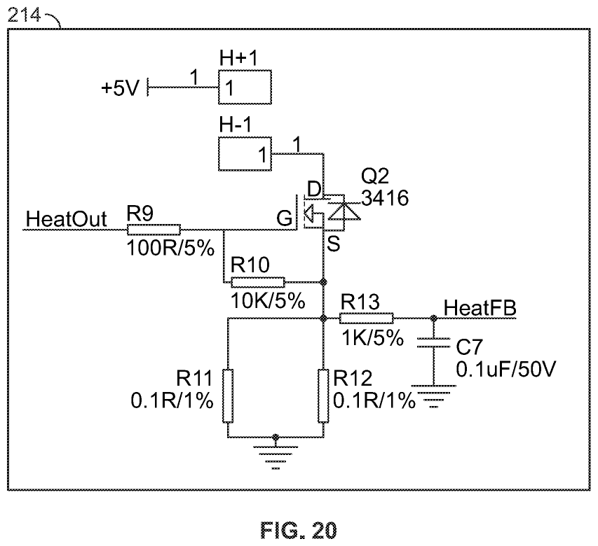

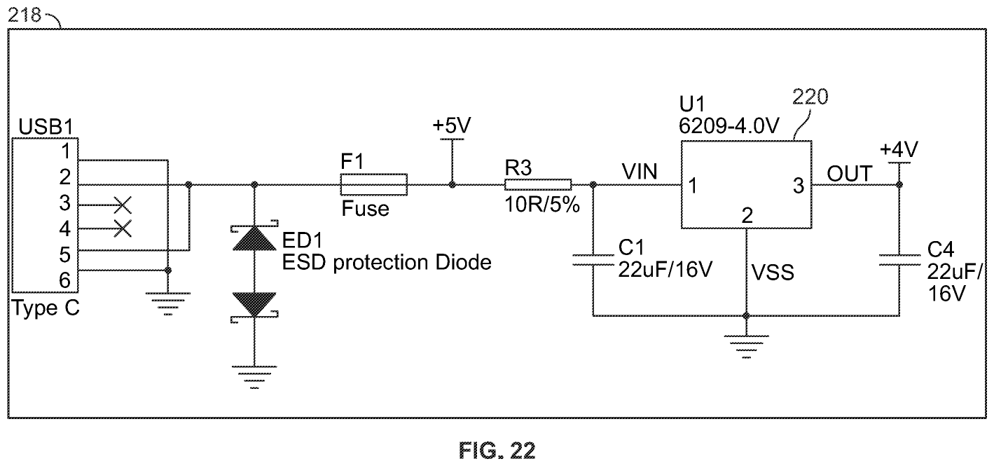

Referring now to FIG. 20, the heater stage 214 is shown in detail. The heater stage 214 includes a plurality of resistors, however more or fewer resistors are contemplated. Referring to FIG. 21, the control stage 216 is used to control the operation of the heating element 102 and other electrical components such as the LEDs 96 and the air displacement mechanism 98. Referring to FIG. 22, the power stage 218 is shown. The power stage 218 may include a voltage regulator 220 and may also include a number of other electrical components, including capacitors, resistors, inductors, diodes, and so forth. In addition, as shown in FIG. 22, the power stage 218 may also include one or more fuses. In some embodiments, the electrical assembly 210 may include a timer, which may allow the control stage 216 to shut off specific electronic components for a predetermined amount of time. Although a particular implementation is shown in FIG. 21, any number of modifications and variations are possible to provide functionalities as described above, as well as other functionalities.

Referring now to FIG. 23, the control PCB 94 and the electronic communication device 86 are shown schematically. As described above, the control PCB 94 includes a plurality of components disposed thereon, which may include a wireless communication device or receiver 222 that may be a module that supports wireless communication. The control PCB 94 may further include a first regulator and a second regulator, either of which may be a voltage regulator. The wireless communication device 222 may support Bluetooth® Low Energy (BLE) wireless communication, Wi-Fi, or other types of wireless communication. In a preferred embodiment, the wireless communication device 222 includes one or more of onboard crystal oscillators, chip antenna, and/or passive components. The wireless communication device 222 may support a number of peripheral functions, e.g., ADC, timers, counters, PWM, and serial communication protocols, e.g., I2C, UART, SPI, through its programmable architecture. The electronic device 86 may include a processor, a flash memory, a timer, and additional components not specifically noted herein. The electronic device 86 may be used to collect and transmit information to the control PCB 94 such as user preferences, switch selection, and environmental factors. In some embodiments, the electronic device 86 is used to collect environmental factors using environmental sensors.

Referring now to FIG. 24, the controller stage 216 receives information from various information sources which may include environmental sensors 224, user inputs 226, and/or information from a lookup table regarding chemical characteristics 228 from a set of fragrance characteristics. In some embodiments, the environmental sensors 224 may comprise the sensors listed above that may detect the cartridge 130 or the type of volatile material within the cartridge 130. The controller stage 216 may include a processor (not shown) that processes the information to determine an optimal power level for each of the discrete fragrance characteristic switching levels, i.e., an optimal heating temperature can be determined to produce a desired scent. In addition, the controller stage 216 may determine a power level to be sent to the air dispensing device 98 to produce a desired airflow. The environmental sensors 224 that may be used could include, but are not limited to, a microphone, camera, turbidity sensor, thermometer, humidity sensor, passive infrared sensor, light sensor, lightning sensor, wind transducer, compass, GPS, gyroscope, accelerometer, barometer, crash sensor, proximity sensor, radar, ultrasonic sensors, or any combination thereof. The chemical characteristics could be measured by the device or transmitted to the device through the wireless communication device or other means. Some of the user inputs 226 may include but are not limited to time preferences for operation, intensity level, and fragrance selection. The controller stage 216 receives the information from the environmental sensors 224, the user inputs 226, and the chemical characteristics 228 of the volatile, and regulates the dispenser 60 to achieve a desired heating temperature 230 and a desired airflow 232 of the air dispensing device 98.

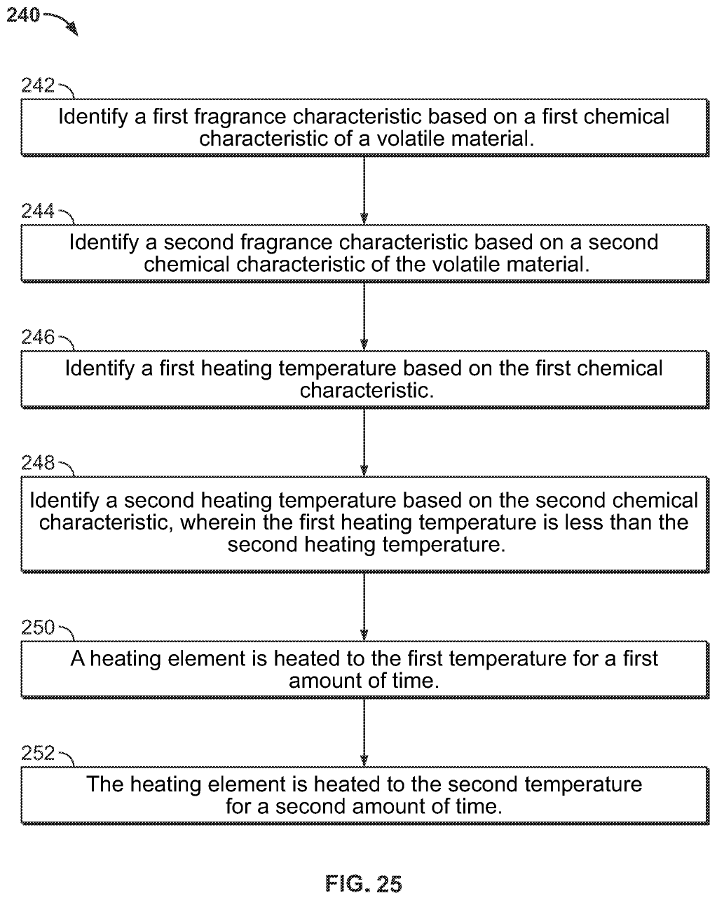

In FIG. 25, a method 240 for emitting the volatile material is shown, which may include more or fewer steps than depicted. At a first step 242, a first fragrance characteristic is identified based on a first chemical characteristic of a volatile material. At second step 244, a second fragrance characteristic is identified based on a second chemical characteristic of the volatile material. At step 246, a first heating temperature is identified based on the first chemical characteristic. At step 248, a second heating temperature is identified based on the second chemical characteristic, wherein the first heating temperature is less than the second heating temperature. At step 250, a heating element is heated to the first temperature for a first amount of time. At step 252, the heating element is heated to the second temperature for a second amount of time.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Any of the embodiments described herein may be modified to include any of the structures or methodologies disclosed in connection with different embodiments. Further, the present disclosure is not limited to the dispensing systems of the type specifically shown. Still further, the methods and systems of any of the embodiments disclosed herein may be modified to work with any type of volatile material dispenser.

### INDUSTRIAL APPLICABILITY

Numerous modifications to the present disclosure will be apparent to those skilled in the art in view of the foregoing description. Accordingly, this description is to be construed as illustrative only and is presented for the purpose of enabling those skilled in the art to make and use the disclosure. The exclusive rights to all modifications which come within the scope of the appended claims are reserved.

1. A method of emitting a volatile material from a volatile material dispenser, comprising the steps of:
identifying a first vapor pressure of a first fragrance characteristic of the volatile material;
identifying a second vapor pressure of a second fragrance characteristic of the volatile material that is different than the first vapor pressure;
heating the volatile material to a first temperature associated with the first fragrance characteristic; and
heating the volatile material to a second temperature associated with the second fragrance characteristic that is different than the first temperature.

2. The method of claim 1 further including the step of:
causing air to flow out of the volatile material dispenser by an air displacement mechanism.

3. The method of claim 2, wherein the air displacement mechanism is an air pump.

4. The method of claim 2, wherein the air displacement mechanism is a fan.

5. The method of claim 4 further comprising the step of:
controlling a speed of the air displacement mechanism to achieve varied fragrance intensities.

6. The method of claim 1 further comprising the step of:
heating the volatile material to the first temperature and the second temperature using pulse with modulation.

7. The method of claim 1 further comprising the step of:
heating the volatile material to the first temperature for a first amount of time and the second temperature to a second amount of time that is different than the first amount of time.

8. The method of claim 1, wherein the first fragrance characteristic is associated with a first composition that is not a fragrance, and the second fragrance characteristic is associated with a second composition that is a fragrance.

9. A method of emitting a volatile material from a first material dispenser, comprising the steps of:
providing a refill containing a volatile material;
heating the volatile material to a first temperature associated with a first fragrance characteristic;
heating the volatile material to a second temperature associated with a second fragrance characteristic; and
heating the volatile material to a third temperature associated with a third fragrance characteristic,
wherein the first temperature is less than the second temperature, and the second temperature is less than the first temperature, and
wherein the first fragrance characteristic, the second fragrance characteristic, and the third fragrance characteristic are different from one another.

10. The method of claim 9, wherein the first fragrance characteristic, the second fragrance characteristic, and the third fragrance characteristic are identified based on one or more chemical characteristics of the volatile material.

11. The method of claim 10, wherein the one or more chemical characteristics includes a vapor pressure.

12. The method of claim 9, wherein the first fragrance characteristic is based on a first perfume raw material, the second fragrance characteristic is based on a second perfume raw material, and the third fragrance characteristic is based on a third perfume raw material, and
wherein the first perfume raw material, the second perfume raw material, and the third perfume raw material are different from one another.

13. The method of claim 10, wherein the first fragrance characteristic is associated with a first wattage, the second fragrance characteristic is associated with a second wattage, and the third fragrance characteristic is associated with a third wattage, and
wherein the first wattage, the second wattage, and the third wattage are different from one another.

14. The method of claim 13, wherein the first wattage, the second wattage, and the third wattage are between about 4.0 W and about 6.0 W.

15. The method of claim 13, wherein the first wattage, the second wattage, and the third wattage are between about 1.0 W and about 9.0 W.

16. A method for emitting a volatile material within a refill from a volatile material dispenser, the method comprising:
heating the volatile material to a first temperature;
controlling an intensity of a fragrance of the volatile material using a first switch;
controlling a fragrance characteristic of the volatile material using a second switch that is configured to adjust the heat applied to the volatile material to a second temperature that is different than the first temperature; and
receiving information from one or more information sources to regulate the volatile material dispenser between the first temperature and the second temperature.

17. The method of claim 16, wherein the first switch has a first switch setting associated with a first timing between each operational cycle, a second switch setting associated with a second timing between each operational cycle, and a third switch setting associated with a third timing between each operational cycle, and
wherein the first timing, the second timing, and the third timing are different from one another.

18. The method of claim 16, wherein the second switch has a first switch setting defining a first fragrance characteristic, a second switch setting defining a second fragrance characteristic, and a third switch setting defining a third fragrance characteristic,
wherein the first fragrance characteristic is associated with a first power input, the second fragrance characteristic is associated with a second power input, and the third fragrance characteristic is associated with a third power input, and
wherein the first power input, the second power input, and the third power input are different from one another.

19. The method of claim 16 further including a third switch that is a push button on/off switch.

20. The method of claim 16, wherein the first switch and the second switch are provided via a display screen of an electronic device that is in communication with electrical components of the volatile material dispenser.
